(19) United States
(12) Patent Application Publication
(10) Pub. No.: US 2018/0375809 A1
(43) Pub. Date: Dec. 27, 2018

(54) EMOTION-SENSING SYSTEM AND METHOD

(71) Applicant: Kit Yi LO, Fo Tan (HK)

(72) Inventor: Kit Yi LO, Fo Tan (HK)

(21) Appl. No.: 16/015,038

(22) Filed: Jun. 21, 2018

(30) Foreign Application Priority Data
Jun. 22, 2017 (CN) 201710481838.0

(51) Int. Cl. H04L 12/58; G06K 9/00; A61B 5/024; G06F 3/0346

(52) U.S. Cl. CPC H04L 51/08; G06F 3/0346; A61B 5/02427; G06K 9/00335

(57) ABSTRACT

An emotion-sensing system comprises a data processing device. The data processing device comprises a communication module, a processing module, an analysis module, a calculation module and an emotional status generation module. The communication module is used for receiving a plurality of photoplethysmographic signals of the user. The processing module is used for processing the plurality of photoplethysmographic signals to obtain a plurality of pulse-to-pulse interval data. The analysis module is used for analyzing the plurality of pulse-to-pulse interval data to obtain different power values and different power average values. The calculation module is used for calculating a pressure level and an emotion value based on the different power values or/and the different power average values respectively. The emotional status generation module is used for comparing the emotion value and the pressure level with the demographic data to deteil line the emotional status of the user.

### CROSS-REFERENCE TO RELATED APPLICATION

This application claims the priority benefits of China Patent Application No. 201710481838.0, filed on Jun. 22, 2017. The entirety the above-mentioned patent application is hereby incorporated by reference herein and made a part of this specification.

### BACKGROUND OF THE INVENTION

### 1. Field of the Invention

The present disclosure generally relates to an emotion-sensing system and a method thereof, and more particularly to an emotion-sensing system and a method for sensing an emotional status according to photoplethysmographic signals.

### 2. Description of Related Art

Modern people are increasingly focusing on their personal health, and many health management devices have become essential products for such people. The common health management device is mostly used for measuring physiological parameters, such as heartbeat, pulse, respiration, and the walking steps of the user, such that a user may check his or her physiological health status via these physiological parameters. In addition to the physiological status, the user's psychological status may not be ignored. If the user experiences a high psychological pressure for a long time, the user's mental health may be influenced. The original life routine of the user may be disturbed by insomnia or emotional instability, and even the user's physiological health may be seriously influenced. Therefore, some health management devices have begun to integrate the related functions of emotion sensing, and the physiological parameters measured by the health management device are used as the standard for detei nining the emotional status. When the user checks the physiological health status by the health management device, the psychological status may be checked at the same time to help the user to prepare appropriate measures early when the emotional status of the user is not good.

However, when the traditional health management device determines the emotional status according to physiological parameters of the user, the accuracy of the emotion sensing is often influenced by extrinsic factors, such that the differences between the sensing result of the emotional status and the real emotion may occur. The desire of the user to buy similar devices will be decreased. Accordingly, there is a need to improve the traditional emotion-sensing system.

### SUMMARY OF THE INVENTION

An object of this disclosure is to provide an emotion-sensing system and a method for sensing and calculating photoplethysmographic signals of a user to deten tine an emotional status of the user so as to improve the accuracy of the emotion sensing and reduce the costs.

To achieve the aforesaid and other objects, the emotion-sensing system of this disclosure comprises a data processing device. The data processing device comprises a communication module, a storage module, a processing module, an analysis module, a calculation module and an emotional status generation module. The communication module is used for receiving a plurality of photoplethysmographic signals of the user. The storage module is used for storing demographic data. The processing module is used for processing the plurality of photoplethysmographic signals to obtain a plurality of pulse-to-pulse interval data. The analysis module is used for analyzing the plurality of pulse-to-pulse interval data to obtain a plurality of high-frequency power values, a plurality of low-frequency power values, a high-frequency power average value and a low-frequency power average value. The calculation module is used for calculating a pressure level based on a last high-frequency power value and a last low-frequency power value and for calculating an emotion value based on the last high-frequency power value, the last low-frequency power value, the high-frequency power average value and the low-frequency power average value. The emotional status generation module is used for comparing the emotion value and the pressure level with the demographic data to determine the emotional status of the user.

The emotion-sensing method of this disclosure comprises the following steps: obtaining a plurality of photoplethysmographic signals of the user; processing the plurality of photoplethysmographic signals to obtain a plurality of pulse-to-pulse interval data; analyzing the plurality of pulse-to-pulse interval data to obtain a plurality of high-frequency power values, a plurality of low-frequency power values, a high-frequency power average value and a low-frequency power average value; calculating a pressure level based on a latest high-frequency power value and a latest low-frequency power value and calculating an emotion value based on the latest high-frequency power value, the latest low-frequency power value, the high-frequency power average value and the low-frequency power average value; and comparing the emotion value and the pressure level with demographic data to determine the emotional status of the user.

### BRIEF DESCRIPTION OF THE DRAWINGS

The accompanying drawings are included to provide a further understanding of the invention and are incorporated in and constitute a part of this specification. The drawings illustrate embodiments of the invention and, together with the descriptions, serve to explain the principles of the invention.

FIG. 1 illustrates use of the emotion-sensing system of this disclosure;

FIG. 2 illustrates a block diagram of the emotion-sensing system of this disclosure;

FIG. 3 illustrates a flowchart of the emotion-sensing method of this disclosure.

### DESCRIPTION OF THE EMBODIMENTS

Since various aspects and embodiments are merely exemplary and not limiting, after reading this specification, those skilled in the art will appreciate that other aspects and embodiments are possible without departing from the scope of the disclosure. Other features and benefits of any one or more of the embodiments will be apparent from the following detailed description and the claims.

The indefinite articles “a” or “an” are employed to describe elements and components described herein merely for convenience and to give a general sense of the scope of the disclosure. Accordingly, this description should be read to include one or at least one and the singular also includes the plural unless it is obvious that it is meant otherwise.

As used herein, the teims “comprises,” “comprising,” “includes,” “including,” “has,” “having” or any other variations thereof are intended to cover a non-exclusive inclusion. For example, a component, structure, article, or apparatus that comprises a list of elements is not necessarily limited to only those elements but may include other elements not expressly listed or inherent to such component, structure, article, or apparatus.

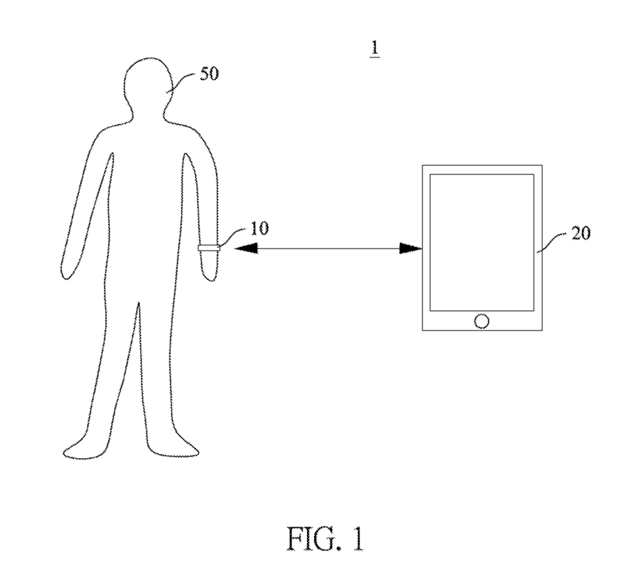

Please refer to FIG. 1, which illustrates use of the emotion-sensing system of this disclosure. As illustrated in FIG. 1, the emotion-sensing system 1 of this disclosure is used for sensing an emotional status of a user 50. The emotion-sensing system comprises a wearable device 10 and a data processing device 20. The data are transmittable between the wearable device 10 and the data processing device 20 via a wireless network or other transmission protocols. In one embodiment of this disclosure, the wearable device 10 is wearable on a body of the user 50 and is a device such as a watch, a wristband or other structures. The data processing device 20 may be a mobile communication device, such as a smart phone.

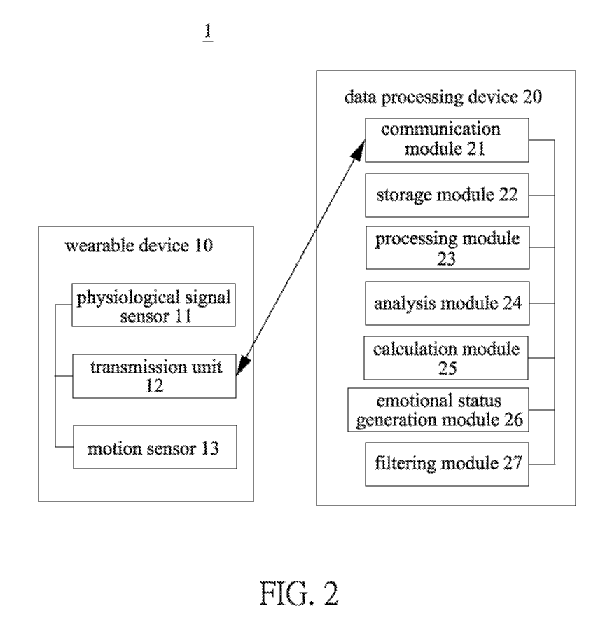

Now refer to FIG. 2, which illustrates a block diagram of the emotion-sensing system of this disclosure. As illustrated in FIG. 2, the wearable device 10 comprises a physiological signal sensor 11 and a transmission unit 12. The physiological signal sensor 11 is used for sensing physiological signals of the user. In this disclosure, the physiological signal sensor 11 is a photoplethysmographic (PPG) sensor for sensing photoplethysmographic signals of the user. The photoplethysmographic sensor uses a photoelectric method to detect blood in a blood vessel via a transmitted light or a scattered light so as to obtain pulse blood change wavefoinis and transfer the waveforms of the photoplethysmographic signals (such as the photoplethysmographic signals corresponding to Hex format data). The transmission unit 12 is electrically connected with the physiological signal sensor 11. The transmission unit 12 is used for transmitting the photoplethysmographic signals to the data processing device 20, such that the photoplethysmographic signals will be the primary factor for determining the emotional status of the user. The transmission unit 12 may be a Bluetooth transmission unit.

Moreover, in one embodiment of this disclosure, the wearable device 10 further comprises a motion sensor 13. The motion sensor 13 is electrically connected with the transmission unit 12. The motion sensor 13 is used for sensing a real-time motion status of the user to obtain a motion status signal, and the motion status signal is also transmitted by the transmission unit 12 to the data processing device 20.

The data processing device 20 comprises a communication module 21, a storage module 22, a processing module 23, an analysis module 24, a calculation module 25 and an emotional status generation module 26, and the aforesaid modules are electrically connected with one another. The communication module 21 is used for receiving the photoplethysmographic signals transmitted from the transmission unit 12 of the wearable device 10. The communication module 21 may be a module that supports wireless communication. The storage module 22 is used for storing demographic data. The storage module 22 may be a memory, a hard disk, or other hardware or firmware or combination thereof capable of storing data. The demographic data is a comparison reference data of the emotional status census of the population data under different conditions; for example, the emotional data are collected from the majority of a population in different condition ranges of age, gender, or other demographic infoitnation, such that the data distributions of the emotion value and/or the pressure level corresponding to each condition range are obtained to be employed as reference data for determining the user's emotional state.

The processing module 23 is used for processing the plurality of photoplethysmographic signals to obtain a plurality of pulse-to-pulse interval (PPI) data. The analysis module 24 is used for analyzing the plurality of pulse-to-pulse interval data to obtain a plurality of high-frequency power values and a plurality of low-frequency power values. A high-frequency power average value is obtained by calculating the plurality of high-frequency power values, and a low-frequency power average value is obtained by calculating the plurality of low-frequency power values. The calculation module 25 is used for importing a last high-frequency power value of the plurality of high-frequency power values and a last low-frequency power value of the plurality of low-frequency power values into specific formulas to calculate a pressure level, and for importing the last high-frequency power value, the last low-frequency power value, the high-frequency power average value and the low-frequency power average value into the specific formulas to calculate an emotion value. The emotional status generation module 26 is used for comparing the emotion value and the pressure level with the demographic data to determine the emotional status of the user. The processing module 23, the analysis module 24, the calculation module 25 and the emotional status generation module 26 may be a portion of an application respectively, which is perfon ied by a processor of the data processing device 20 to achieve corresponding functions, but this disclosure is not limited thereto. Each aforesaid module may also be a combination of hardware and fimiware or software (such as an operating system).

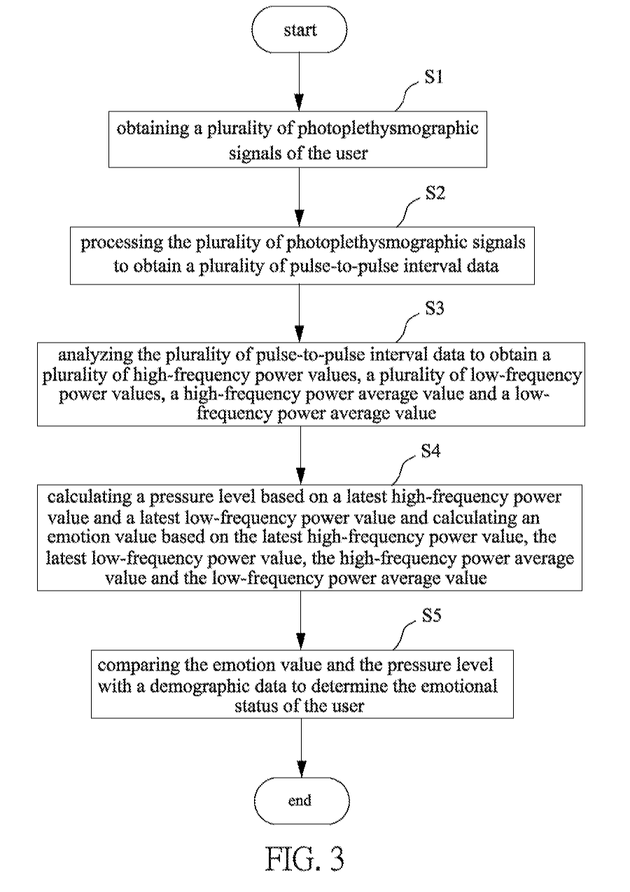

Please refer to FIG. 3, which illustrates a flowchart of the emotion-sensing method of this disclosure. It should be noted that the emotion-sensing method of this disclosure is explained by the emotion-sensing system 1 of FIG. 2 as an example, but this disclosure is not limited thereto. As illustrated in FIG. 3, the emotion-sensing method of this disclosure comprises Steps S1 to S5, which are exemplified in detail below.

Step S1: obtaining a plurality of photoplethysmographic signals of the user.

First, the photoplethysmographic signals of the user are sensed by the physiological signal sensor 11. The photoplethysmographic signals of the user are sensed continuously by the physiological signal sensor 11 and transmitted sequentially to the data processing device 20. For the designs of the disclosure, the photoplethysmographic signals of the user are sensed continuously by the physiological signal sensor 11 in a time period, such that the plurality of photoplethysmographic signals are received by the communication module 21 in the time period. In one embodiment of this disclosure, a time period of about 3 to 5 minutes is sufficient to collect enough photoplethysmographic signals to be the references for calculation and comparison, but the time period is not limited thereto. Accordingly, in this embodiment, a plurality of continuous or non-continuous time periods (for example, an idle time is between two adjacent time periods) are divided from a longer time period (such as a day or an hour) by the emotion-sensing system 1 and are used for sensing continuously or intermittently by the physiological signal sensor 11.

Step S2: processing the plurality of photoplethysmographic signals to obtain a plurality of pulse-to-pulse interval data.

After the plurality of photoplethysmographic signals of the user are obtained in Step S1, the processing module 23 processes each photoplethysmographic signal to obtain each corresponding pulse-to-pulse interval (PPI) data until the amount of the pulse-to-pulse interval data is equal to the number of the photoplethysmographic signals.

Step S3: analyzing the plurality of pulse-to-pulse interval data to obtain a plurality of high-frequency power values, a plurality of low-frequency power values, a high-frequency power average value and a low-frequency power average value.

After a plurality of pulse-to-pulse interval data are obtained in Step S2, the analysis module 24 analyzes each pulse-to-pulse interval data to obtain each corresponding high-frequency power value and each corresponding low-frequency power value until the number of the high-frequency power values and low-frequency power values are equal respectively to the amount of the pulse-to-pulse interval data. Then the high-frequency power average value is obtained by calculating the plurality of high-frequency power values, and the low-frequency power average value is obtained by calculating the plurality of low-frequency power values. Accordingly, if a greater number of high-frequency power values and low-frequency power values are obtained, a larger database can be formed. Therefore, the high-frequency power average value and low-frequency power average value which have been calculated reflect the real status of the sensed data.

To ensure that the high-frequency power values and low-frequency power values are effective data and to reduce errors, in one embodiment of this disclosure, the data processing device 20 further comprises a filtering module 27 for filtering an outlier from the high-frequency power value or the low-frequency power value. For example, if the high-frequency power value or the low-frequency power value is zero, it is an outlier. Accordingly, when the filtering module 27 determines that the high-frequency power value or the low-frequency power value is zero, the pulse-to-pulse interval data corresponding to the high-frequency power value or the low-frequency power value is further analyzed.

If the pulse-to-pulse interval data corresponding to the high-frequency power value or the low-frequency power value is more than a maximum threshold (for example, the real PPI data is more than 4000 ms and represents a number of heartbeats of the user of less than 15) to detemiine that an outlier exists, the filtering module 27 selects a last high-frequency power value or a last low-frequency power value instead of the high-frequency power value or the low-frequency power value. Furthermore, if the pulse-to-pulse interval data corresponding to the high-frequency power value or the low-frequency power value is less than a minimum threshold (for example, the real PPI data is less than 250 ms and represents a number of heartbeats of the user of more than 200) to determine that an outlier exists, the filtering module 27 selects to ignore the high-frequency power value or the low-frequency power value. However, the maximum threshold or the minimum threshold is adjustable for different needs or settings.

Moreover, when the filtering module 27 determines that a frequency of occurrence of the outlier in a preset time period has reached a threshold, it means that the user is not wearing the wearable device properly or that the user's body on which the wearable device is worn is moving so fast as to cause an error in the data sensing. Therefore, the filtering module 27 requests the processing module 23 to execute a resetting process to re-sense and obtain the photoplethysmographic signals of the user.

When the frequency of occurrence of outliers reaches or exceeds the threshold because of the aforesaid conditions of the user, the number of the effective high-frequency power values or the effective low-frequency power values which are obtained in the preset time period will be decreased, and this decrease will influence the high-frequency average power value or the low-frequency average power value which is calculated such that the accuracy of the emotional status which is determined will be degraded. Therefore, by executing the resetting process, the processing module 23 will clear all of the pulse-to-pulse interval data, the high-frequency power values and the low-frequency power values in the preset time period, and the processing module 23 will restart to receive and process new photoplethysmographic signals in another preset time period to obtain new high-frequency power values and new low-frequency power values based on the new photoplethysmographic signals. The influence of the excessive outliers of the high-frequency power values and the low-frequency power values may thus be avoided.

Step S4: calculating a pressure level based on a latest high-frequency power value and a latest low-frequency power value and calculating an emotion value based on the latest high-frequency power value, the latest low-frequency power value, the high-frequency power average value and the low-frequency power average value.

After the high-frequency power values, the low-frequency power values, the high-frequency power average value and the low-frequency power average value are obtained in Step S3, the calculation module 25 calculates the pressure level and the emotion value via the specific different foil iulas respectively for determining the emotional status of the user. The last high-frequency power value of the plurality of high-frequency power values and the last low-frequency power value of the plurality of low-frequency power values are imported into the specific formulas to calculate a pressure level corresponding to the last high-frequency power value and the last low-frequency power value. Furthermore, the last high-frequency power value, the last low-frequency power value, the high-frequency power average value and the low-frequency power average value are imported into the specific formulas to calculate an emotion value corresponding to the last high-frequency power value and the last low-frequency power value.

Step S5: comparing the emotion value and the pressure level with demographic data to determine the emotional status of the user.

After calculating the pressure level and the emotion value of the user in Step S4, the emotional status generation module 26 selects suitable demographic data and compares the emotion value and the pressure level with the suitable demographic data to determine the emotional status of the user.

In one embodiment of this disclosure, the storage module 22 further comprises a personal setting value of the user. The personal setting value is capable of being inputted and stored in the storage module 22 by the user before the emotion-sensing method of this disclosure is perfoitited. The personal setting value comprises age, sex, nationality or other standard data. In Step S5, the emotional status generation module 26 selects the demographic data in line with the personal setting value, such that the demographic data is closer to the real conditions of the user to improve the accuracy of the determined emotional status. For example, if the inputted age of the personal setting value of the user is 35 years old, the demographic data corresponds to two different ranges of 0 to 50 years old and 51 to 100 years old respectively. Accordingly, the emotional status generation module 26 selects the demographic data corresponding to the range of 0 to 50 years old according to the personal setting value of the user as the reference standard for the pressure level and the emotion value. The corresponding range, sub-range or point of the demographic data to which the pressure level and the emotion value belong is used to determine the corresponding emotional status of the user.

The demographic data are capable of being stored in the storage module 22 in advance or being downloaded or updated from an external server by the emotional status generation module 26 transmitting a download request to an external server.

Accordingly, the emotion-sensing method of this disclosure is used for analyzing and processing the photoplethysmographic signals and calculating the pressure level and the emotion value based on the corresponding high-frequency power values and average power value and the corresponding low-frequency power values and average power value and for determining the emotional status of the user according to the demographic data. The accuracy of the emotional status is improved and the costs of calculation during the process are reduced. Moreover, in one embodiment of this disclosure, the final emotional status is contributorily determined according to a motion status signal of the real-time motion status of the user sensed by the motion sensor 13 of the wearable device 10. When the result of comparing the emotion value and the pressure level of the user with the demographic data is uncertain or falls between two emotional statuses, the emotion value or the pressure level is increased or decreased by contributorily consulting the motion status signal of the user to improve the accuracy of the emotional status. For example, if the comparison of the emotion value and the pressure level of the user with the demographic data indicates a result between happiness and sadness, depending on the degree of change or fluctuation of the motion status signal, the result may reflect that the user is happy and therefore moving his or her hands or that the user feels sad and remains motionless. Therefore, the emotion value or the pressure level is adjustable according to the motion status signal to contributorily determine the emotional status.

The emotional status determined by the emotion-sensing method of this disclosure is combinable with other data or applications to perform different functions. For example, in one embodiment of this disclosure, the storage module 22 further comprises at least one friend's information. The friend's infoimation may be an instant messaging account (such as Facebook, Line, QQ or other such accounts), a telephone number, an email address or other data of the friend. The at least one friend's infoll iation may be the information of one single friend or a plurality of friends in one friend's group. The at least one friend's information is capable of being inputted and stored in the storage module 22 by the user before the emotion-sensing method of this disclosure is performed, or a friends list or a group members list of the instant messaging account can be used directly. When the emotional status generation module 26 determines the emotional status of the user, the emotional status generation module 26 requests the communication module 21 to send an emotion-associated message to another data processing device corresponding to the at least one friend's information according to the emotional status of the user so as to instantly share the emotional status of the user with the friend. The emotion-associated message may comprise an ideogram, a word, a voice or an image corresponding to the emotional status. For example, when the emotional status is happiness, the ideogram for directly or indirectly reflecting the emotional status, such as a smiley face or a sun, or a passage of music or a specific sound, may be transmitted, but this disclosure is not limited thereto. The emotion-associated message may be a pre-set message of the emotional status generation module 26 or a message set by user.

Oppositely, when the friend of the user sends a response message corresponding to the emotion-associated message by the another data processing device, the data processing device 20 receives the response message via the communication module 21 and shows the response message by an output component (such as a display screen) of the data processing device 20 for interactive communication.

In addition to the aforesaid embodiments, the emotional status generation module 26 further actively produces a feedback message in response to the emotional status of the user. For example, in one embodiment of this disclosure, when the emotional status generation module 26 determines the emotional status of the user, the emotional status generation module 26 produces the feedback message according to the emotional status and shows the feedback message by an output component (such as a display screen or an amplifier) of the data processing device 20. The feedback message comprises an ideogram, a word, a voice or an image corresponding to the emotional status, and the feedback message is not directly related to the emotional status. For example, when the emotional status is happiness, the emotional status generation module 26 may produce a smiley face or a passage of relaxed music as the feedback message, and when the emotional status is unhappiness, the emotional status generation module 26 may produce a crying face, an encouragement ideogram or a word as the feedback message, but this disclosure is not limited thereto.

Moreover, the emotional status generation module 26 further links the emotional status and the time sensed the emotional status together and stores the aforesaid data in the storage module 22 to foiin an emotion journal. It is convenient for the user to find the emotional status of the corresponding time.

The above detailed description is merely illustrative in nature and is not intended to limit the embodiments of the subject matter or the application and uses of such embodiments. Moreover, while at least one exemplary embodiment has been presented in the foregoing detailed description, it should be appreciated that a vast number of variations exist. It should also be appreciated that the exemplary one or more embodiments described herein are not intended to limit the scope, applicability, or configuration of the claimed subject matter in any way. Rather, the foregoing detailed description will provide those skilled in the art with a convenient guide for implementing the described one or more embodiments. Also, various changes can be made to the function and arrangement of elements without departing from the scope defined by the claims, which include known equivalents and foreseeable equivalents at the time of filing of this patent application.

What is claimed is:

1. An emotion-sensing system for sensing an emotional status of a user, the emotion-sensing system comprising:
a data processing device, comprising:
a communication module for receiving a plurality of photoplethysmographic signals of the user;
a storage module for storing demographic data;
a processing module for processing the plurality of photoplethysmographic signals to obtain a plurality of pulse-to-pulse interval data;
an analysis module for analyzing the plurality of pulse-to-pulse interval data to obtain a plurality of high-frequency power values, a plurality of low-frequency power values, a high-frequency power average value and a low-frequency power average value;
a calculation module for calculating a pressure level based on a last high-frequency power value and a last low-frequency power value and calculating an emotion value based on the last high-frequency power value, the last low-frequency power value, the high-frequency power average value and the low-frequency power average value; and
an emotional status generation module for comparing the emotion value and the pressure level with the demographic data to determine the emotional status of the user.

2. The emotion-sensing system of claim 1, wherein the plurality of photoplethysmographic signals are received sequentially by the communication module in a time period.

3. The emotion-sensing system of claim 1, further comprising a wearable device, the wearable device comprising:
a physiological signal sensor for sensing the photoplethysmographic signals of the user; and
a transmission unit for transmitting the photoplethysmographic signals to the data processing device.

4. The emotion-sensing system of claim 3, wherein the wearable device further comprises a motion sensor for sensing a real-time motion status of the user, such that the processing module of the data processing device contributorily determines the emotional status of the user according to the real-time motion status.

5. The emotion-sensing system of claim 1, wherein the storage module further comprises a personal setting value of the user, and the emotional status generation module selects the demographic data in line with the personal setting value.

6. The emotion-sensing system of claim 1, wherein the storage module further comprises at least one friend's infoli lation, and the emotional status generation module requests the communication module to send an emotion-associated message to another data processing device corresponding to the at least one friend's information according to the emotional status of the user.

7. The emotion-sensing system of claim 6, wherein the emotion-associated message comprises an ideogram, a voice or an image corresponding to the emotional status.

8. The emotion-sensing system of claim 1, wherein the data processing device further comprises a filtering module for filtering an outlier from the high-frequency power value or the low-frequency power value.

9. The emotion-sensing system of claim 8, wherein when the filtering module determines that a frequency of occurrence of the outlier in a preset time period has reached a threshold, the filtering module requests the processing module to execute a resetting process.

10. An emotion-sensing method for sensing an emotional status of a user, comprising:
obtaining a plurality of photoplethysmographic signals of the user;
processing the plurality of photoplethysmographic signals to obtain a plurality of pulse-to-pulse interval data;
analyzing the plurality of pulse-to-pulse interval data to obtain a plurality of high-frequency power values, a plurality of low-frequency power values, a high-frequency power average value and a low-frequency power average value;
calculating a pressure level based on a latest high-frequency power value and a latest low-frequency power value and calculating an emotion value based on the latest high-frequency power value, the latest low-frequency power value, the high-frequency power average value and the low-frequency power average value; and
comparing the emotion value and the pressure level with demographic data to determine the emotional status of the user.
